(19) United States
(12) Patent Application Publication
(10) Pub. No.: US 2026/0048175 A1
(43) Pub. Date: Feb. 19, 2026

(54) THREE-DIMENSIONAL SCAFFOLD COMPOSITIONS AND METHODS FOR BONE REPAIR OR REGENERATION

(71) Applicant: Rutgers, The State University of New Jersey, New Brunswick, NJ (US)

(72) Inventors: Joseph Warren Freeman, Piscataway, NJ (US); Shreya Madhavarapu, Flemington, NJ (US); Adhithi Kanthan Lakshmikanthan, Irvin, CA (US)

(21) Appl. No.: 19/300,767

(22) Filed: Aug. 15, 2025

Related U.S. Application Data
(60) Provisional application No. 63/683,467, filed on Aug. 15, 2024.

(51) Int. Cl. A61L 27/18; A61L 27/12; A61L 27/36; A61L 27/38; B33Y 80/00; C08L 67/04

(52) U.S. Cl. CPC A61L 27/18; A61L 27/12; A61L 27/3633; A61L 27/3687; A61L 27/3691; A61L 27/3808; A61L 27/3834; B33Y 80/00; C08L 67/04

(57) ABSTRACT

Described herein are three-dimensional scaffold compositions and methods for bone repair or regeneration. In some embodiments, the disclosed scaffolds comprise a core portion mimicking a native trabecular bone structure that is surrounded by an exterior portion mimicking a native cortical bone structure. In some embodiments, the scaffolds may be functionalized by mineralization and/or pre-vascularization to promote bone and blood vessel formation.

### CROSS-REFERENCE TO RELATED APPLICATIONS

This application claims priority to U.S. Provisional Ser. No. 63/683,467 , filed on Aug. 15, 2024, which is incorporated by reference herein in its entirety.

### BACKGROUND

Various bone diseases including bone injury and degeneration are a growing problem worldwide. A main challenge facing bone repair and regeneration is the difficulty of constructing devices with dual structural and functional organization similar to that of natural bone tissue. Two types of structures are present in bones, cortical and trabecular. The former is generally found surrounding the latter. Cortical bone is composed of highly compacted osteons which are oriented parallel to the longitudinal axis of the bone. The channel-like structure inside osteons provides a protecting space, also known as haversian canal, for the growth of vasculature and nerves. The high tensile and mechanical strength of the structure also prevents micro-crack propagation. Surrounded by cortical bone, trabecular bone has weaker mechanical strength but features an extensive interconnected network of pores which may vary significantly.

Different technologies and devices have been developed for bone repair and regeneration. However, many of them still have serious drawbacks. Autograft, a standard approach in orthopedic surgeries, is limited by supply and donor site morbidity. Although an alternative procedure using allograft overcomes the drawbacks of autograft, it still relates to issues such as more incidences of disease transmission and higher failure rate. Synthetic materials made of metal are strong but do not degrade or bond to surrounding bone. Ceramic based bone grafts, although capable of promoting bone growth, are brittle and lack mechanical strength. More recently, scaffolds have been developed for application in bone tissue engineering. However, these scaffolds do not have dual structural organization as found in natural bone tissue, and they also lack the required mechanical strength necessary for load-bearing applications and the necessary cues to promote the regeneration of bone vasculature.

What is needed are novel biocompatible scaffolds for bone repair or regeneration that mimic the structural architecture of native bone for effective tissue growth upon implantation in a subject.

### SUMMARY

One embodiment described herein is a three-dimensional scaffold for bone repair or regeneration comprising: a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion. In one aspect, the interconnected network of spaces forms from about 45% to about 55% of the volume of the core portion. In another aspect, each strut in each layer has a width ranging from about 0.4 mm to about 0.6 mm, wherein each space positioned between adjacent struts in each layer has a width ranging from about 0.4 mm to about 0.6 mm. In another aspect, the core portion and the exterior portion are each independently formed of a material comprising at least one biocompatible polymer selected from the group consisting of a polycarbonate, a polymethylmethacrylate, a polyethylene, a polyurethane, a polyaryl etherketone, a polyetherether-ketone, a polylactic acid (PLA), a polylactide, a polyglycolide, a poly(D, L-lactide), a poly(L-lactide), a poly(glycolide), a poly(s-caprolactone), a poly(dioxanone), a poly(glyconate), a poly(hydroxybutyrate), a poly(hydroxyvalerate), a poly(orthoester), a poly(carboxylate), a poly(propylene fumarate), a poly(phosphate), a poly(anhydride), a poly(iminocarbonate), a poly(phosphazene), and copolymers thereof. In another aspect, the core portion and the exterior portion are each formed of a material comprising a polylactic acid (PLA). In another aspect, the exterior portion has an exterior surface, wherein the surfaces of the struts and the exterior surface of the exterior portion are coated with a calcium phosphate-based mineral. In another aspect, the calcium phosphate-based mineral comprises hydroxyapatite, alpha tricalcium phosphate, beta tricalcium phosphate, or combinations thereof. In another aspect, one or more of the channels of the exterior portion are at least partially filled with a pro-angiogenic extracellular matrix. In another aspect, each of the channels of the exterior portion has a cross-sectional width ranging from about 0.25 mm to about 2.0 mm. In another aspect, each of the channels of the exterior portion has a cross-sectional width ranging from about 0.5 mm to about 1.0 mm. In another aspect, each of the channels of the exterior portion is spaced apart from adjacent channels by about 0.5 mm to about 1.0 mm. In another aspect, the core portion has a diameter ranging from about 3 mm to about 24 mm. In another aspect, the scaffold has a diameter ranging from about 5 mm to about 35 mm. In another aspect, a length of the scaffold from the first end to the second end ranges from about 5 mm to about 500 mm. In another aspect, the scaffold has a compressive modulus ranging from about 400 MPa to about 700 MPa. In another aspect, the scaffold has a compressive yield strength ranging from about 20 MPa to about 50 MPa. In another aspect, the scaffold further comprises stem cells. In another aspect, the stem cells are mesenchymal stem cells (MSCs). In another aspect, the scaffold is three dimensionally printed.

Another embodiment described herein is a method of making a three-dimensional scaffold for bone repair or regeneration, the method comprising: fabricating the three-dimensional scaffold comprising: a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion; coating the surfaces of the struts and an exterior surface of the exterior portion with a calcium phosphate-based mineral to mineralize the scaffold; seeding and culturing cells in one or more of the channels of the exterior portion for a period of time sufficient to produce a pro-angiogenic extracellular matrix that at least partially fills the one or more channels; and removing the cells from the one or more channels of the exterior portion. In one aspect, fabricating the three-dimensional scaffold comprises three-dimensionally printing the scaffold. In another aspect, the calcium phosphate-based mineral comprises hydroxyapatite, alpha tricalcium phosphate, beta tricalcium phosphate, or combinations thereof. In another aspect, the cells comprise endothelial cells. In another aspect, the method further comprises sterilizing the scaffold. In another aspect, the method further comprises freeze-drying the scaffold. In another aspect, the method further comprises adding stem cells to the scaffold. In another aspect, the method further comprises adding autologous bone marrow to the scaffold.

Another embodiment described herein is a method of repairing or regenerating bone in a subject, the method comprising implanting any of the scaffolds described herein into a site in need of bone repair or regeneration in the subject.

Another embodiment described herein is a kit for repairing or regenerating bone in a subject, the kit comprising: any of the scaffolds described herein; and optionally, one or more of packaging or instructions for use.

This disclosure provides for other aspects and embodiments that will be apparent in light of the following detailed description and accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an exemplary process for generating a three-dimensional polymer scaffold as described herein, including three-dimensional (3D) printing of a polylactic acid (PLA) scaffold, mineralization using concentrated simulated body fluid (SBF), and pre-vascularization by culturing human microvascular endothelial cells (HMEC-1) within the cortical sections of the scaffold followed by decellularization (figure Created with BioRender.com).

FIG. 2 shows a top view of an example scaffold as described herein that includes a trabecular core portion with a cortical exterior portion surrounding the circumference of the trabecular core portion.

FIG. 3A-B show example designs for the trabecular core and cortical exterior portions of the scaffold. FIG. 3A shows computer-aided design (CAD) drawings of an example core portion (top) and the orthogonal arrangement of struts between adjacent layers within the core portion (bottom). In this non-limiting example, each strut is about 0.5 mm wide and is spaced apart from adjacent structs by about 0.5 mm. FIG. 3B shows an example exterior portion design having a grid-like structure with a plurality of hollow channels that run along the length of the scaffold, mimicking the osteons of native cortical bone. In this non-limiting example, each hollow channel has a cross-sectional width of about 0.5 or 1 mm and is spaced apart from adjacent channels by about 0.5 or 1 mm.

FIG. 4A-D show the measured mechanical properties, including compressive modulus (CM) (FIG. 4A and C) and compressive yield strength (CYS) (FIG. 4B and D), for different example scaffold designs. FIG. 4A-B show a comparison of the mechanical properties between a trabecular-only scaffold design and two different cortical-only scaffold designs. FIG. 4C-D show a comparison of the mechanical properties between two different complete scaffold designs (Designs 1 and 2) having both trabecular core and cortical exterior portions. Design 2 had larger hollow channels in its cortical exterior portion and exhibited better CM and CYS mechanical properties as compared to Design 1. * p-value<0.05.

FIG. 5A-F show the ability of three different scaffold designs to promote capillary action (i.e., wicking action) at viscosities of 3 cP (FIGS. 5A), 38 cP (FIGS. 5B), 130 cP (FIGS. 5C), 235 cP (FIG. 5D), and 400 cP (FIG. 5E). FIG. 5F shows a top view of the three different scaffolds (shown left to right in each figure) that included a trabecular-only scaffold, a combination “complete” scaffold including both trabecular and cortical portions, and a cortical-only scaffold.

FIG. 6A-D show mineralization data for different electrospun scaffolds produced using a polylactic acid (PLA) polymer material. FIG. 6A shows ELISA data for osteocalcin production after scaffold mineralization over 10 days. FIG. 6B shows the normalized alkaline phosphatase (ALP) area after scaffold mineralization over 10 days. FIG. 6C shows Alizarin red staining for mineral content over 10 days for the same samples tested in FIG. 6B (scale bar=200 μm). FIG. 6D shows a mineral ash weight analysis comparing scaffold surface pretreatment techniques (prior to mineralization). Similar results are expected and have been observed with 3D-printed scaffolds made from the same PLA polymer material.

FIG. 7A-C show pre-vascularization data for different electrospun scaffolds produced using a polylactic acid (PLA) polymer material. FIG. 7A shows ELISA data for vascular endothelial growth factor (VEGF) concentration produced from seeded hMSCs after scaffold pre-vascularization over 12 days. FIGS. 7B and 7C show VE cadherin and CD31 staining, respectively, for hMSCs seeded in a pre-vascularized scaffold or a non-vascularized scaffold over 12 days. Similar results are expected and have been observed with 3D-printed scaffolds made from the same PLA polymer material.

FIG. 8A-D show results of bone regeneration using a New Zealand white rabbit critical-sized radial defect model over 20 weeks with a mineralized and pre-vascularized scaffold including a trabecular core portion and a cortical exterior portion surrounding its circumference. FIG. 8A (top) shows X-ray images taken at the 2-week, 4-week, 6-week, 8-week, and 10-week timepoints after the radial defect surgery. FIG. 8A (bottom) shows live animal CT scan images at the 12-week, 16-week, and 20-week timepoints showing bone growth into and within the scaffold, and a micro-CT scan image (post excision) at the end of the study showing formation of mature bone tissue. FIG. 8B shows a reconstructed micro-CT scan image showing clear bone infiltration and mature bone formation within the scaffold implant. FIG. 8C shows the volume of new bone growth throughout the study as determined from the 3D reconstructed CT scan images at the 12-week, 16-week, and 20-week timepoints. FIG. 8D shows histological H&E staining analysis showing bone regeneration within the polymer scaffold implant along with the formation of blood vessels (shown with black arrowheads).

FIG. 9 shows histological staining of unseeded scaffolds. Mineralized bone tissue appeared dark pink (H&E), blue (MT), and red (VVG). Arrows indicate blood vessels. Scale bar=60 μm.

FIG. 10 shows histological staining of bone marrow seeded scaffolds. Mineralized bone tissue appeared dark pink (H&E), blue (MT), and red (VVG). Arrows indicate blood vessels or signs of vasculature. Scale bar=60 μm.

FIG. 11 shows histological staining of allograph samples. Mineralized bone tissue appeared dark pink (H&E), blue (MT), and red (VVG). Arrows indicate blood vessels or signs of vasculature. Scale bar=60 μm.

FIG. 12 shows immunological staining of unseeded scaffolds (Group 1), bone marrow seeded scaffolds (Group 2), or allographs (Group 3) for anti-CD31+DAPI and anti-vWF+DAPI. The images show CD31 and vWF staining indicating blood vessels.

FIG. 13A-B show quantification of immunological staining of CD31 (in FIG. 12). The percentage of stained area was calculated and compared across the three groups, with analyses performed on sections from the middle of the implant (A) and the ends (B). * Indicates statistical significance with p<0.05. Seeded scaffolds had the largest amount of CD 31 (not significant) at the ends of the implant. Allografts had the largest amount in the middle.

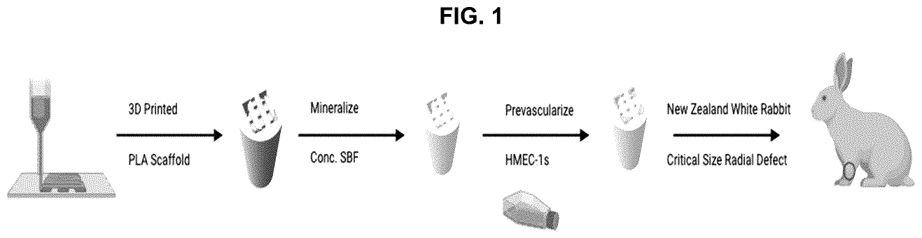

Before any embodiments of this disclosure are explained in detail, it is to be understood that the disclosure is not limited in its application to the details of construction and the arrangement of components set forth in the following description or illustrated in the accompanying figures. Also, it is to be understood that the phraseology and terminology used herein is for the purpose of description and should not be regarded as limiting.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,”“an,”or “the”means “one or more”unless otherwise specified.

As used herein, the term “or”can be conjunctive or disjunctive.

As used herein, the term “and/or”refers to both the conjunctive and disjunctive.

As used herein, the term “substantially” means to a great or significant extent, but not completely. As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo-and ribo-nucleotides, and combinations of bases including uracil, adenine, thymine, cytosine, guanine, inosine, xanthine, hypoxanthine, isocytosine and isoguanine. Nucleic acids may be obtained by chemical synthesis methods or by recombinant methods.

As used herein, the terms “active ingredient” or “active pharmaceutical ingredient” refer to a pharmaceutical agent, active ingredient, compound, cell, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, or nanoparticles.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In one embodiment, the agent or composition may be provided in a powder form and mixed with a liquid, such as water, to form a beverage. In accordance with the present disclosure, “administering” can be self-administering. For example, it is considered “administering” when a subject consumes a composition as disclosed herein.

As used herein, the terms “effective amount” or “therapeutically effective amount,” refers to a substantially non-toxic, but sufficient amount of an action, agent, composition, scaffold, or cell(s) being administered to a subject that will prevent, treat, or ameliorate to some extent one or more of the symptoms of the disease, injury, or condition being experienced or that the subject is susceptible to contracting. The result can be the reduction or alleviation of the signs, symptoms, or causes of a disease or injury, or any other desired alteration of a biological system. An effective amount may be based on factors individual to each subject, including, but not limited to, the subject's age, size, type or extent/severity of disease or injury, stage of the disease or injury, route of administration, the type or extent of supplemental therapy used, ongoing disease or injury process, and the type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. As used herein, a subject is “in need of treatment” if such subject would benefit biologically, medically, or in quality of life from such treatment. A subject in need of treatment does not necessarily present symptoms, particularly in the case of preventative or prophylaxis treatments. In some non-limiting embodiments of the present disclosure, a subject is in need of treatment if the subject is suffering from a bone injury or bone defect, such as a crushed or shattered bone.

As used herein, the term “endogenous” refers to any material from or produced inside an organism, cell, tissue, or system.

As used herein, the term “exogenous” refers to any material introduced from or produced outside an organism, cell, tissue, or system.

As used herein, the terms “inhibit,” “inhibition,” or “inhibiting” refer to the reduction or suppression of a given biological process, condition, symptom, disorder, injury, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder, injury, or disease, or completely eliminating a disease or injury. A treatment may be either performed in an acute or chronic manner. The term “treatment” also refers to reducing the severity of an injury or disease or symptoms associated with such disease prior to affliction with the disease. “Repressing” or “ameliorating” an injury, disease, disorder, or the symptoms thereof involves administering a cell, composition, scaffold, or compound described herein to a subject after clinical appearance of such injury, disease, disorder, or its symptoms. “Prophylaxis of” or “preventing” an injury, disease, disorder, or the symptoms thereof involves administering a cell, composition, scaffold, or compound described herein to a subject prior to onset of the injury, disease, disorder, or the symptoms thereof. “Suppressing” an injury, disease, or disorder involves administering a cell, composition, scaffold, or compound described herein to a subject after induction of the injury, disease, or disorder thereof but before its clinical appearance or symptoms thereof have manifested.

Described herein are 3D scaffold compositions and methods that provide an improved approach for bone repair or regeneration as compared to the standard of care. For example, the disclosed 3D scaffolds provide the advantage of being load-sharing scaffolds that mimic native bone trabecular and cortical architectures, and exhibit sustained mechanical stability in vivo. The scaffolds facilitate infiltration of bone marrow, reducing the need to add stem cells, and promote vascularized bone regeneration and tissue growth without the use of additional growth factors.

In certain non-limiting exemplary embodiments described herein, the disclosed 3D scaffolds are designed to promote new bone growth and blood vessel innervation using isolated stem cells from a subject, stem cells within the subject's bone marrow, or cells from the surrounding tissue. The 3D scaffolds mimic the normal bone architecture with a highly porous inner portion (trabecular bone) and a more compact surrounding outer portion (cortical bone). The scaffolds can then be coated with a fine layer of calcium phosphate-based mineral within the trabecular portion and around the cortical portion (i.e., mineralization) to promote osteogenic differentiation, and further seeded with vascular cells to produce pro-angiogenic extracellular matrix within the scaffold (i.e., pre-vascularization) that promote the conversion of infiltrated stem cells into vascular endothelial cells. The scaffolds are then decellularized, sterilized (e.g., using ethylene oxide gas), and ready for implantation. Three-dimensional bone scaffolds, mineralization processes, and pre-vascularization processes have been described in U.S. Pat. No. 10,524,915, the entire contents of which are hereby incorporated into the specification.

One embodiment described herein is a three-dimensional scaffold for bone repair or regeneration comprising: a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion. In one aspect, the interconnected network of spaces forms from about 45% to about 55% of the volume of the core portion. In some embodiments, additional pores may be introduced into the core portion and/or exterior portion of the scaffold using, for example, a soak-freeze technique.

In another aspect, each strut in each layer has a width ranging from about 0.4 mm to about 0.6 mm, wherein each space positioned between adjacent struts in each layer has a width ranging from about 0.4 mm to about 0.6 mm. These sizes and dimensions are exemplary and not meant to be limiting. For example, disclosed struts may have a width less than 0.4 mm or greater than 0.6 mm, and each space positioned between adjacent struts may have a width less than 0.4 mm or greater than 0.6 mm.

In another aspect, the core portion and the exterior portion are each independently formed of a material comprising at least one biocompatible polymer selected from the group consisting of a polycarbonate, a polymethylmethacrylate, a polyethylene, a polyurethane, a polyaryl etherketone, a polyetherether-ketone, a polylactic acid (PLA), a polylactide, a polyglycolide, a poly(D, L-lactide), a poly(L-lactide), a poly(glycolide), a poly(ϵ-caprolactone), a poly(dioxanone), a poly(glyconate), a poly(hydroxybutyrate), a poly(hydroxyvalerate), a poly(orthoester), a poly(carboxylate), a poly(propylene fumarate), a poly(phosphate), a poly(anhydride), a poly(iminocarbonate), a poly(phosphazene), and copolymers thereof. In another aspect, the core portion and the exterior portion are each formed of a material comprising a polylactic acid (PLA). In some embodiments, additional compounds or materials that enhance desirable mechanical and/or biological properties may also be added to the scaffold composition.

In some embodiments described herein, the disclosed polymer scaffolds exhibit negligible degradation and no reduction in mechanical strength over 20 weeks following implantation in vivo. It is possible to control or modulate the rate of polymer degradation based on the molecular weight of the polymer. For example, the degradation rate can be increased (made quicker) by reducing the molecular weight of the polymer or decreased (made slower) by increasing the molecular weight. In some non-limiting exemplary embodiments, the biocompatible polymer used to form the different scaffold portions may have a molecular weight ranging from about 50 kDa to about 150 kDa.

In some embodiments, the scaffold may be further mineralized. Mineralization comprises incubating the scaffold in a solution of one or more salts at room temperature or at an elevated temperature. Non-limiting examples of salts include NaCl, KCl, CaCl, MgCl₂, and NaH₂PO₄. The time of incubation can be about 30 minutes, about 1 hour, about 2 hours, about 5 hours, about 12 hours, about 16 hours, about 24 hours, about 36 hours, about 48 hours, or more than 48 hours. Mineralization processes can be important for promoting osteogenesis or the conversion of stem cells into osteoblasts upon scaffold implantation.

Various buffering agents may also be included in the solution for mineralization. Buffering agents suitable for use with the present disclosure include, for example, phosphates, such as sodium phosphate; phosphates monobasic, such as sodium dihydrogen phosphate and potassium dihydrogen phosphate; phosphates dibasic, such as disodium hydrogen phosphate and dipotassium hydrogen phosphate; citrates, such as sodium citrate (anhydrous or dehydrate); and bicarbonates, such as sodium bicarbonate and potassium bicarbonate that may be used. In some embodiments, a single buffering agent, e.g., a dibasic buffering agent is used. In some embodiments, a combination of buffering agents is employed, e.g., a combination of a tri-basic buffering agent and a monobasic buffering agent. In one aspect, the exterior portion of the scaffold has an exterior surface, wherein the surfaces of the struts and the exterior surface of the exterior portion are coated with a calcium phosphate-based mineral. In another aspect, the calcium phosphate-based mineral comprises hydroxyapatite, alpha tricalcium phosphate, beta tricalcium phosphate, or combinations thereof.

In another aspect, one or more of the channels of the exterior portion are at least partially filled with a pro-angiogenic extracellular matrix that has been decellularized (i.e., pre-vascularization). In some non-limiting embodiments, the pro-angiogenic extracellular matrix may be formed using cultured endothelial vascular cells including, but not limited to, human microvascular endothelial cells (HMEC-1) or human umbilical vein endothelial cells (HUVECs), that are then removed through decellularization to make the scaffold cell-free and non-immunogenic. This pre-vascularization process can be important for the conversion of stem cells into vascular endothelial cells upon scaffold implantation.

In another aspect, each of the channels of the exterior portion has a cross-sectional width ranging from about 0.25 mm to about 2.0 mm. In another aspect, each of the channels of the exterior portion has a cross-sectional width ranging from about 0.5 mm to about 1.0 mm. In another aspect, each of the channels of the exterior portion is spaced apart from adjacent channels by about 0.5 mm to about 1.0 mm. In another aspect, the core portion has a diameter ranging from about 3 mm to about 24 mm. In another aspect, the scaffold has a diameter ranging from about 5 mm to about 35 mm. In another aspect, a length of the scaffold from the first end to the second end ranges from about 5 mm to about 500 mm. All of these disclosed sizes and dimensions are exemplary and not meant to be limiting. The specific shape and size dimensions of the described scaffolds will vary depending on, for example, the particular application, subject age and condition, specific bone type and size, bone injury or defect site, degree/severity of bone injury or defect, etc. For example, disclosed scaffolds may have diameters less than 5 mm or greater than 35 mm, and may have lengths less than 5 mm or greater than 500 mm.

The disclosed scaffolds can exhibit mechanical properties similar to those of native bone. For example, in one aspect, the scaffold has a compressive modulus ranging from about 400 MPa to about 700 MPa. In another aspect, the scaffold has a compressive yield strength ranging from about 20 MPa to about 50 MPa.

In another aspect, the scaffold further comprises stem cells. Stem cells can be incorporated into the scaffold for differentiating into multiple cellular lineages. Various types of stem cells are reported in the literature for tissue engineering; see e.g., Li et al., Stem Cells Transl. Med. 2(9): 667-677 (2013). Stem cells suitable for use with the disclosed scaffolds, including for example muscle-derived stem cells (MDSCs), adipose derived stem cells, and mesenchymal stem cells (MSCs), can be allogeneic or native. In one aspect, the stem cells are mesenchymal stem cells (MSCs). Stem cells can be populated throughout the scaffold, including the hollow channels of the exterior cortical portion and the spaces of the trabecular core portion, or in a particular section of the scaffold.

In another aspect, the scaffold is three dimensionally printed using known techniques and commercially available instruments including, but not limited to, Ultimaker fused deposition modeling (FDM) 3D printers. The specific sizes and dimensions of the trabecular core portion and the cortical exterior portion of the described scaffolds can be adjusted and determined based on what the native trabecular to cortical bone ratio is at a particular defect site in a subject. For example, in some embodiments, the disclosed scaffolds can be custom-printed based on the specific defect, where a CT scan or other known technique may be used to determine the specific size and shape of the scaffold. In other embodiments, the disclosed scaffolds can be readily available in different shapes and sizes as an off-the-shelf option. For an off-the-shelf option, scaffolds can be stored at −80° C. or freeze-dried like other commercially available grafts (e.g., decellularized allografts).

Another embodiment described herein is a method of making a three-dimensional scaffold for bone repair or regeneration, the method comprising: fabricating the three-dimensional scaffold comprising: a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion; coating the surfaces of the struts and an exterior surface of the exterior portion with a calcium phosphate-based mineral to mineralize the scaffold; seeding and culturing cells in one or more of the channels of the exterior portion for a period of time sufficient to produce a pro-angiogenic extracellular matrix that at least partially fills the one or more channels; and removing the cells from the one or more channels of the exterior portion. In one aspect, fabricating the three-dimensional scaffold comprises three-dimensionally printing the scaffold. In another aspect, the calcium phosphate-based mineral comprises hydroxyapatite, alpha tricalcium phosphate, beta tricalcium phosphate, or combinations thereof. In another aspect, the cells comprise endothelial cells. In another aspect, the method further comprises sterilizing the scaffold. In another aspect, the method further comprises freeze-drying the scaffold. In another aspect, the method further comprises adding stem cells to the scaffold. In another aspect, the method further comprises adding autologous bone marrow to the scaffold.

Another embodiment described herein is a method of repairing or regenerating bone in a subject, the method comprising implanting any of the scaffolds described herein into a site in need of bone repair or regeneration in the subject, such as a bone defect or bone injury site. In some embodiments, the method may comprise creating a subcutaneous pocket on a subject at the site in need of bone repair or regeneration and placing the scaffold into the pocket.

In some embodiments, the method may further comprise the use of additional hardware in combination with the scaffolds disclosed herein to support the repair or regeneration of bone. For example, the scaffolds described herein are strong enough to partially bear physiological loads, so they can be used with an inert, metallic implant, or in conjunction with an intramedullary nail (rod) along with a minimal number of pins or plates. In other embodiments, it may be possible to use the disclosed scaffolds without additional hardware being present in the site in need of bone repair or regeneration if one or more other bones is intact and can provide initial support. In some embodiments, it may also be possible to use these scaffolds without hardware in non-load bearing applications such as in repairing craniofacial bones and other small bones (e.g., fingers). Another embodiment described herein is a kit for repairing or regenerating bone in a subject, the kit comprising: any of the scaffolds described herein; and optionally, one or more of packaging or instructions for use.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A three-dimensional scaffold for bone repair or regeneration comprising:
a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and
an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion.

Clause 2. The scaffold of clause 1, wherein the interconnected network of spaces forms from about 45% to about 55% of the volume of the core portion.

Clause 3. The scaffold of clause 1 or 2, wherein each strut in each layer has a width ranging from about 0.4 mm to about 0.6 mm, and wherein each space positioned between adjacent struts in each layer has a width ranging from about 0.4 mm to about 0.6 mm.

Clause 4. The scaffold of any one of clauses 1-3, wherein the core portion and the exterior portion are each independently formed of a material comprising at least one biocompatible polymer selected from the group consisting of a polycarbonate, a polymethylmethacrylate, a polyethylene, a polyurethane, a polyaryl etherketone, a polyetherether-ketone, a polylactic acid (PLA), a polylactide, a polyglycolide, a poly(D, L-lactide), a poly(L-lactide), a poly(glycolide), a poly(s-caprolactone), a poly(dioxanone), a poly(glyconate), a poly(hydroxybutyrate), a poly(hydroxyvalerate), a poly(orthoester), a poly(carboxylate), a poly(propylene fumarate), a poly(phosphate), a poly(anhydride), a poly(iminocarbonate), a poly(phosphazene), and copolymers thereof.

Clause 5. The scaffold of any one of clauses 1-4, wherein the core portion and the exterior portion are each formed of a material comprising a polylactic acid (PLA).

Clause 6. The scaffold of any one of clauses 1-5, wherein the exterior portion has an exterior surface, and wherein the surfaces of the struts and the exterior surface of the exterior portion are coated with a calcium phosphate-based mineral.

Clause 7. The scaffold of any one of clauses 1-6, wherein the calcium phosphate-based mineral comprises hydroxyapatite, alpha tricalcium phosphate, beta tricalcium phosphate, or combinations thereof.

Clause 8. The scaffold of any one of clauses 1-7, wherein one or more of the channels of the exterior portion are at least partially filled with a pro-angiogenic extracellular matrix.

Clause 9. The scaffold of any one of clauses 1-8, wherein each of the channels of the exterior portion has a cross-sectional width ranging from about 0.25 mm to about 2.0 mm.

Clause 10. The scaffold of any one of clauses 1-9, wherein each of the channels of the exterior portion has a cross-sectional width ranging from about 0.5 mm to about 1.0 mm.

Clause 11. The scaffold of any one of clauses 1-10, wherein each of the channels of the exterior portion is spaced apart from adjacent channels by about 0.5 mm to about 1.0 mm.

Clause 12. The scaffold of any one of clauses 1-11, wherein the core portion has a diameter ranging from about 3 mm to about 24 mm.

Clause 13. The scaffold of any one of clauses 1-12, wherein the scaffold has a diameter ranging from about 5 mm to about 35 mm.

Clause 14. The scaffold of any one of clauses 1-13, wherein a length of the scaffold from the first end to the second end ranges from about 5 mm to about 500 mm.

Clause 15. The scaffold of any one of clauses 1-14, wherein the scaffold has a compressive modulus ranging from about 400 MPa to about 700 MPa.

Clause 16. The scaffold of any one of clauses 1-15, wherein the scaffold has a compressive yield strength ranging from about 20 MPa to about 50 MPa.

Clause 17. The scaffold of any one of clauses 1-16, further comprising stem cells.

Clause 18. The scaffold of any one of clauses 1-17, wherein the stem cells are mesenchymal stem cells (MSCs).

Clause 19. The scaffold of any one of clauses 1-18, wherein the scaffold is three dimensionally printed.

Clause 20. A method of making a three-dimensional scaffold for bone repair or regeneration,
the method comprising:
fabricating the three-dimensional scaffold comprising:
   a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and
   an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion;
coating the surfaces of the struts and an exterior surface of the exterior portion with a calcium phosphate-based mineral to mineralize the scaffold;
seeding and culturing cells in one or more of the channels of the exterior portion for a period of time sufficient to produce a pro-angiogenic extracellular matrix that at least partially fills the one or more channels; and
removing the cells from the one or more channels of the exterior portion.

Clause 21. The method of clause 20, wherein fabricating the three-dimensional scaffold comprises three-dimensionally printing the scaffold.

Clause 22. The method of clause 20 or 21, wherein the calcium phosphate-based mineral comprises hydroxyapatite, alpha tricalcium phosphate, beta tricalcium phosphate, or combinations thereof.

Clause 23. The method of any one of clauses 20-22, wherein the cells comprise endothelial cells.

Clause 24. The method of any one of clauses 20-23, further comprising sterilizing the scaffold.

Clause 25. The method of any one of clauses 20-24, further comprising freeze-drying the scaffold.

Clause 26. The method of any one of clauses 20-25, further comprising adding stem cells to the scaffold.

Clause 27. The method of any one of clauses 20-26, further comprising adding autologous bone marrow to the scaffold.

Clause 28. A method of repairing or regenerating bone in a subject, the method comprising implanting the scaffold of any one of clauses 1-19 into a site in need of bone repair or regeneration in the subject.

Clause 29. A kit for repairing or regenerating bone in a subject, the kit comprising:
the scaffold of any one of clauses 1-19; and
optionally, one or more of packaging or instructions for use.

### EXAMPLES

### Example 1

### Design and Fabrication of 3D Scaffolds

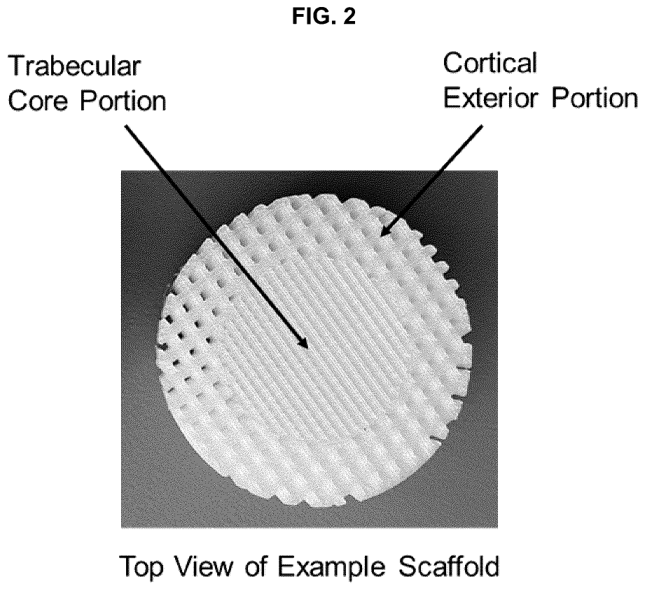

Scaffolds were designed using the computer-aided design (CAD) software SolidWorks (Dassault Systèmes) with emphasis on porosity and pore size. The scaffolds were designed to mimic the natural trabecular and cortical architectures of native bone. The central trabecular core section of the scaffold was designed with alternating orthogonal horizontal and vertical layers. FIG. 2 shows a top view of an example 3D-printed scaffold that includes a trabecular core portion with a cortical exterior portion surrounding its circumference.

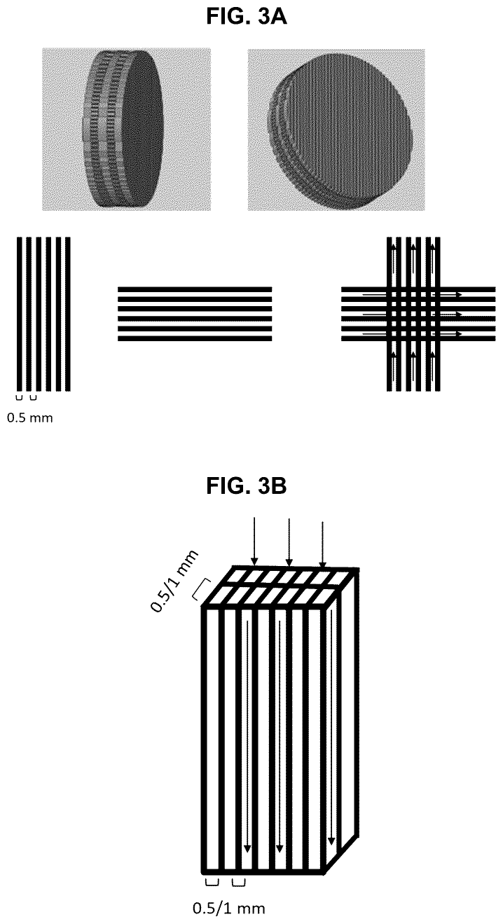

FIG. 3A-B show example designs for the trabecular core and cortical exterior portions of the scaffold. FIG. 3A shows CAD drawings of an example core portion (top) and the orthogonal arrangement of the struts between adjacent layers within the core portion (bottom). The trabecular core portion has a mesh-like or porous design with multiple layers of orthogonal struts that enable nutrient flow in horizontal and vertical directions. In the example configuration shown in FIG. 3A, each strut has a width of about 0.5 mm and is spaced apart from adjacent struts by about 0.5 mm. This generates an interconnected network of spaces (i.e., “pores”) extending through the multiple layers of orthogonal struts, with the interconnected network of spaces forming from about 40% to about 60% of the volume of the trabecular core portion.

FIG. 3B then shows an example cortical exterior portion design having a grid-like structure with a plurality of hollow cylindrical channels that run along the length of the scaffold, mimicking the osteons of native cortical bone. In the example configuration shown in FIG. 3B, the cross-sectional dimensions of the hollow channels are either about 0.5 mm×0.5 mm or about 1.0 mm×1.0 mm, and are spaced apart from adjacent channels by about 0.5 mm to about 1.0 mm. These hollow channels typically form from about 20% to about 30% of the volume of the cortical exterior portion.

For 3D printing, Cura software (Ultimaker) was used to define print parameters such as layer height, infill percentage, etc., followed by slicing the designs to generate the gcode. An Ultimaker 2+ 3D printer was used to print the scaffolds with PLA filament purchased from Ultimaker and Gizmo Dorks (2.85 mm filament diameter, MW 60,000-80,000). The scaffolds were printed using a 0.25 mm diameter print nozzle to achieve the best possible resolution.

### Example 2

### Mechanical Characterization of 3D Scaffolds

Compression analysis was performed to determine the mechanical strength properties of different 3D scaffolds. The scaffolds were compressed at a crosshead speed of 1 mm/min (strain rate of 10%/min) using the Instron 5869 mechanical testing system. The generated data were analyzed to determine the compressive modulus (CM) and compressive yield strength (CYS) values.

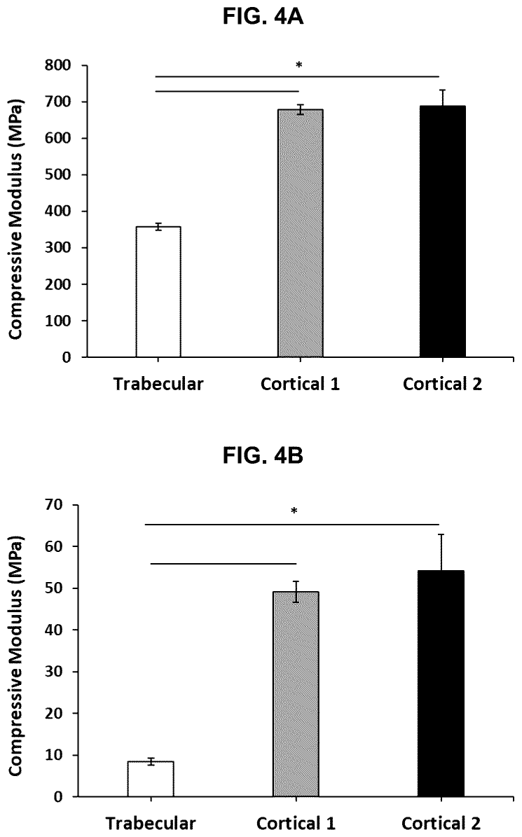

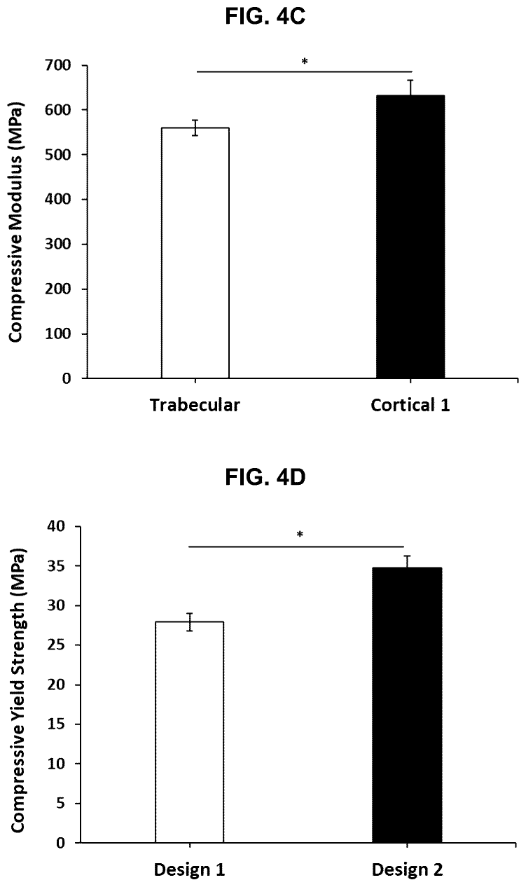

FIG. 4A-B show the measured CM and CYS mechanical properties for different example 3D scaffold designs. A trabecular-only scaffold design was compared to two different cortical-only scaffold designs (FIG. 4A). The trabecular-only scaffold exhibited a CM of 356.21±7.95 MPa and a CYS of 9.24±0.41 MPa (FIG. 4A), which are similar values to those of native trabecular bone (CM of 7-200 MPa, CYS of 0.2-10 MPa). FIG. 4B shows a comparison of the CM and CYS mechanical properties between two different complete scaffold designs having both trabecular core and cortical exterior portions (Designs 1 and 2). Design 1 had cortical exterior channels with dimensions of 0.5 mm×0.5 mm, while Design 2 had larger cortical exterior channels with dimensions of 1.0 mm×1.0 mm. Designs 1 and 2 displayed CM values of 563.22±12.80 and 632.43±32.06 MPa, and CYS values of 28.06±0.93 and 34.74±1.67 MPa, respectively (FIG. 4B). These results demonstrated that Design 2 exhibited better CM and CYS mechanical properties as compared to Design 1.

### Example 3

### Capillary Action of 3D Scaffolds

The ability of the 3D scaffolds to promote capillary action was tested using different glycerol-DI water solutions that mimic the viscosities of blood (3.5-5.5 cP) and bone marrow (37.5-400 cP). The three different types of scaffolds (shown left to right in each figure) were a trabecular-only scaffold, a combination scaffold including both trabecular and cortical portions, and a cortical-only scaffold.

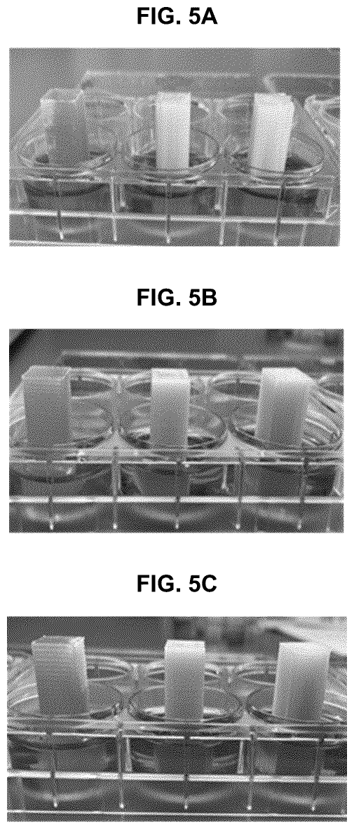

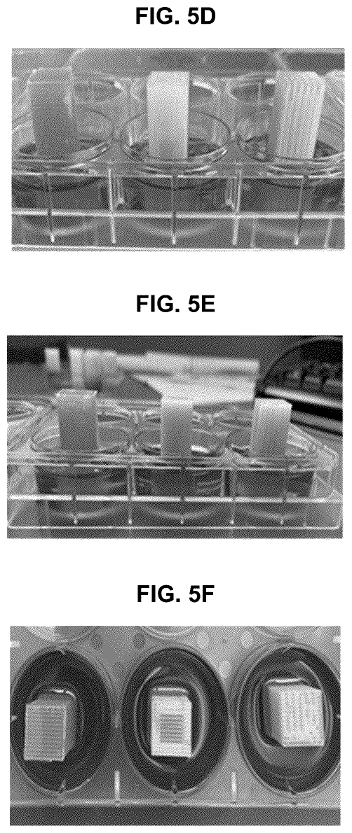

FIG. 5A-F show the ability of the different scaffold designs to promote capillary action (i.e., wicking action). The specific viscosities tested were: 3 cP (FIGS. 5A), 38 cP (FIGS. 5B), 130 cP (FIGS. 5C), 235 cP (FIG. 5D), and 400 cP (FIG. 5E). FIG. 5F shows a top view of the three scaffolds. Capillary action was observed for all three types of scaffolds, demonstrating that the highly porous structures within the scaffolds can promote capillary inflow of blood and bone marrow. The scaffolds displayed the ability to promote the infiltration of bone marrow-like solution into and throughout the scaffold, reducing the need to add stem cells prior to implantation in vivo.

### Example 4

### Mineralization of Scaffolds

Scaffolds were mineralized using concentrated simulated body fluid (10× SBF) solution containing hydroxyapatite (HAP) calcium phosphate-based minerals. For the mineralization process, calcium ions from the SBF attach to nucleation sites on the polymer scaffold surface. SBF has a calcium concentration similar to blood plasma, and the addition of the inorganic content in the form of Ca²⁺ ions improves the scaffold's osteoblastic differentiation capability. Static mineralization of scaffolds was carried out in 10× SBF. The mineralization procedure involved placing scaffolds in vials filled with 100:1 SBF and NaHCO₃ and placing the vials on a shaker. The solutes used in the SBF, sodium chloride (NaCl), potassium chloride (KCl), calcium chloride dihydrate (CaCl₂·H₂O), magnesium chloride heptahydrate (MgCl₂·H₂O), sodium bicarbonate (NaHCO₃), and sodium phosphate monobasic (NaH₂PO₄) were purchased from Fisher Scientific (Pittsburgh, PA, USA). Mineralization was carried out for 20 hours with the 10× SBF solution being changed every 2 hours.

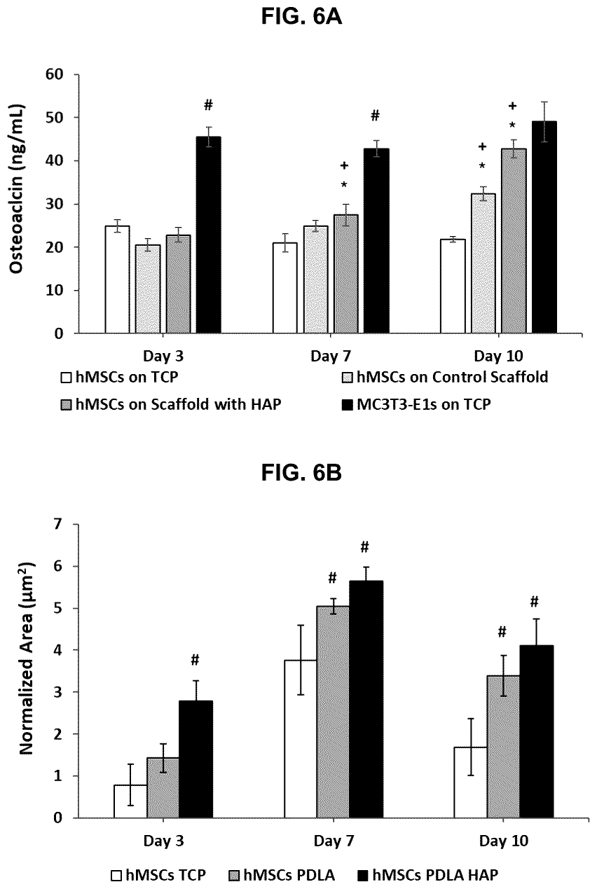

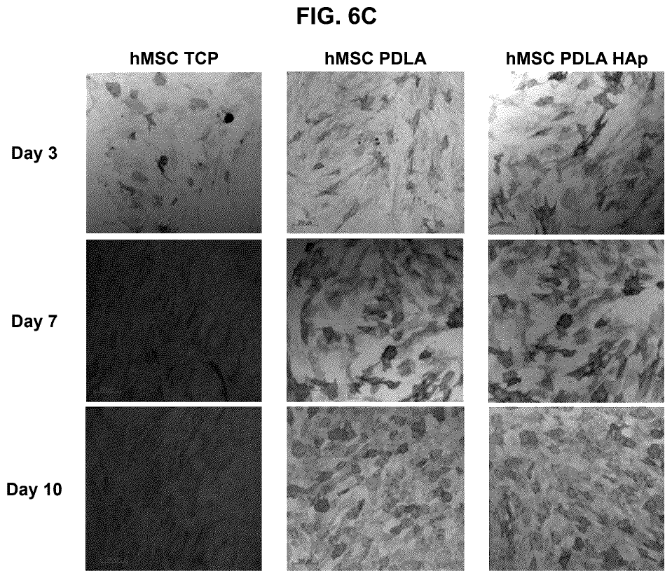

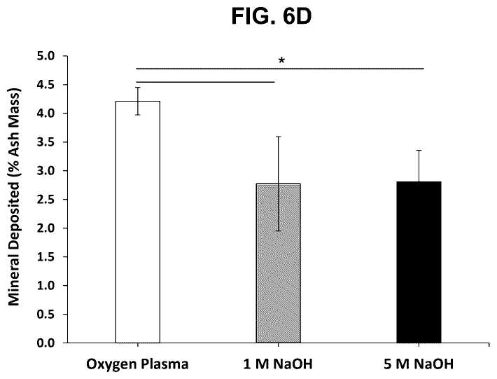

FIG. 6A-D show mineralization data for different electrospun scaffolds produced using a polylactic acid (PLA) polymer material. FIG. 6A shows ELISA data for osteocalcin production after scaffold mineralization over 10 days. Osteocalcin is a non-collagenous protein produced by mature osteoblasts during later stages of differentiation and is a protein crucial to the mineralization of bone and is characteristic of osteoblasts differentiating to osteocytes. The different samples that were tested included human mesenchymal stem cells (hMSCs) seeded on tissue culture plastic (TCP) control, hMSCs seeded on a control scaffold with no mineralization, hMSCs seeded on a scaffold with hydroxyapatite (HAP) mineralization, and a pre-osteoblast control (MC3T3-E1).

The hMSC cells were seeded at passage 3 and a seeding density of 15,000 cells/cm2. Cells used for this study were first grown in osteogenic media-the Mesenchymal Stem Cell Growth Kit, with Mesenchymal Stem Cell Basal Medium, L-Alanyl-L-Glutamine, 30% FBS, rh-IGF, rh-FGF, and 1% Penicillin-Streptomycin (ATCC). When seeding on scaffolds as well as subsequent feeding, the media used was standard osteoblast media without any osteogenic factors—α-MEM (Life Technologies) with 10% FBS and 1% Penicillin-Streptomycin. ELISA assays were performed to study the differentiation of the hMSCs. Media collected was tested for the bone marker osteocalcin. The ELISA kits were purchased from RayBio®. Samples were also fixed at days 4, 8, and 12 and stained for alkaline phosphatase (ALP), an early marker for osteogenesis, using an ALP staining kit (Sigma-Aldrich), and the amount of mineral remaining on the scaffolds at different time points was estimated and compared using Alizarin red staining (Alizarin red powder, Sigma Aldrich). All the images were obtained using a Zeiss confocal microscope.

The hMSCs seeded on mineralized scaffolds produced more osteocalcin than those seeded on unmineralized scaffolds and were comparable to the pre-osteoblast MC3T3-E1 control, as measured by ELISA (FIG. 6A). #, +, * p-value<0.05, n=6.

FIG. 6B shows the normalized ALP area after scaffold mineralization over 10 days. The different samples that were tested included hMSCs seeded on TCP control, hMSCs seeded on a poly(D-lactide) (PDLA) scaffold with no mineralization, and hMSCs seeded on a PDLA scaffold with HAP mineralization. The hMSCs seeded on mineralized scaffolds showed significantly more ALP than those seeded on unmineralized scaffolds. # p-value<0.05, n=6.

FIG. 6C shows Alizarin red staining for mineral content over 10 days for the same samples tested in FIG. 6B (scale bar=200 μm). The hMSCs seeded on mineralized scaffolds showed significantly more Alizarin red stained areas than those seeded on unmineralized scaffolds.

FIG. 6D shows a mineral ash weight analysis comparing scaffold surface pretreatment techniques (prior to mineralization). Pretreatment with oxygen plasma allowed for significantly more mineral to be deposited on the scaffold surface when compared to pretreatment with two different concentrations of NaOH. * p-value<0.05.

Overall, these data indicated that mineralization of the electrospun scaffolds promotes osteogenesis. Similar results are expected and have been observed with 3D-printed scaffolds made from the same PLA polymer material.

### Example 5

### Pre-Vascularization of the Exterior Cortical Portion of Scaffolds

In order to promote angiogenic and vasculogenic differentiation of stem cells once scaffolds are implanted in vivo, the exterior portions (i.e., cortical sections) of the scaffolds surrounding the trabecular core portion were pre-vascularized by seeding with HMEC-1 cells (ATCC). HMEC-1 cells (passage 4-5) were seeded within the cortical sections. The media used for this study was MCDB 131 medium (Life Technologies) with 1 μg/mL hydrocortisone, 10 mM glutamine, 10% FBS, 1% Penicillin-Streptomycin, and epidermal growth factor or EGF (10 ng/ml). Scaffolds and cells on tissue culture plastic (TCP) control were also fixed using 4% paraformaldehyde (PFA, Sigma Aldrich) for CD31 and VE cadherin staining. The remaining scaffolds were decellularized using a freeze-thaw method to remove the HMEC-1 cells and leave behind a pro-angiogenic matrix. Briefly, the scaffolds were first placed in liquid nitrogen for 10 minutes, then in a water bath maintained at 37° C. for 10 minutes. The scaffolds were washed with PBS (3×) before repeating the freeze-thaw cycle 2 more times. Post decellularization, the scaffolds were stored at −80° C.

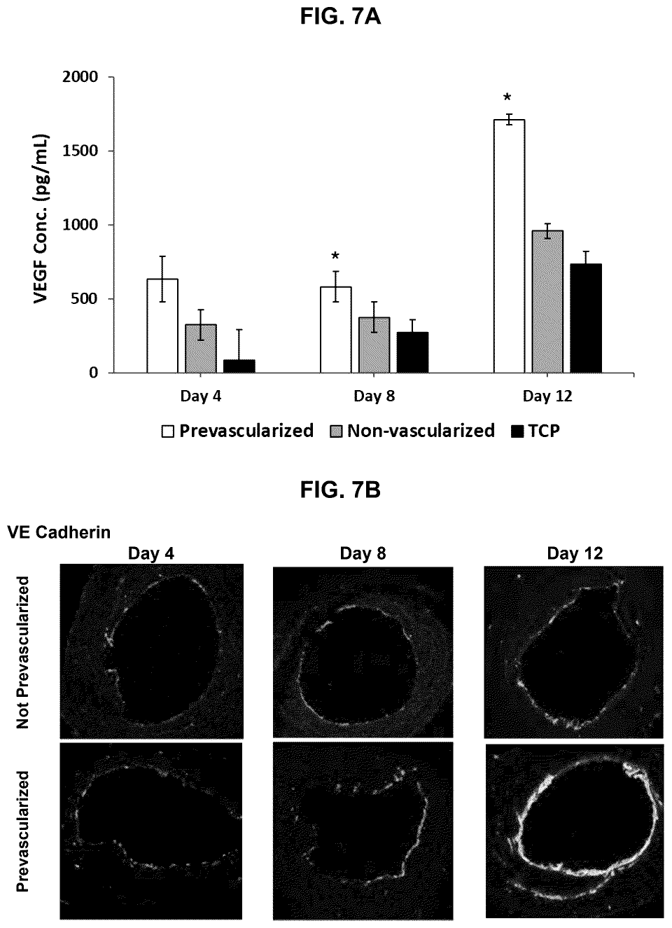

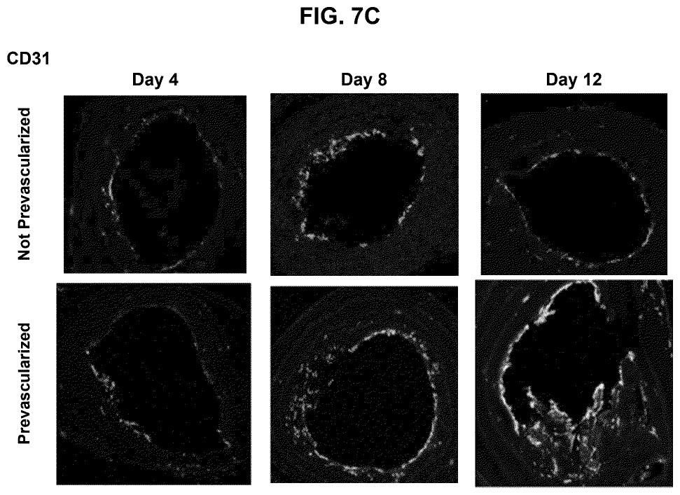

FIG. 7A-C show pre-vascularization data for different electrospun scaffolds produced using a polylactic acid (PLA) polymer material. FIG. 7A shows ELISA data for vascular endothelial growth factor (VEGF) concentration produced from seeded hMSCs after scaffold pre-vascularization over 12 days. VEGF is one of the most prominent angiogenesis stimulatory molecules that induces endothelial cell migration and proliferation. The different samples that were tested included hMSCs seeded in a pre-vascularized mineralized scaffold having a pro-angiogenic matrix within the channels of the cortical exterior portion, hMSCs seeded in a non-vascularized mineralized scaffold having no pro-angiogenic matrix, and hMSCs seeded on TCP control.

The hMSC cells were seeded at passage 3 and a seeding density of 15,000 cells/cm2. Cells used for this study were first grown in osteogenic media-the Mesenchymal Stem Cell Growth kit, with Mesenchymal Stem Cell Basal Medium, L-Alanyl-L-Glutamine, 30% FBS, rh-IGF, rh-FGF, and 1% Penicillin-Streptomycin (ATCC). When seeding on scaffolds as well as subsequent feeding, the media used was standard osteoblast media without any osteogenic factors—α-MEM (Life Technologies) with 10% FBS and 1% Penicillin-Streptomycin. ELISA assays were performed to study the differentiation of the hMSCs. Media collected was tested for the endothelial marker VEGF-A. The ELISA kits were purchased from RayBio®. Samples were also fixed at days 4, 8, and 12 for immunohistochemical staining for VE cadherin and CD31. The VE cadherin and CD31 primary monoclonal antibodies and the secondary antibodies were purchased from Thermo Fisher Scientific. All the images were obtained using a Zeiss confocal microscope. Higher VEGF levels were observed for the pre-vascularized scaffolds than the non-vascularized scaffolds at all timepoints, and the TCP controls produced the least VEGF. At the final 12-day timepoint, it can be observed that pre-vascularized scaffolds with a VEGF concentration of 1731±189 pg/mL significantly outperformed the non-vascularized scaffolds having a VEGF concentration of 977±69 pg/mL (FIG. 7A). * p-value<0.05.

FIGS. 7B and 7C show VE cadherin and CD31 staining, respectively, for hMSCs seeded in a pre-vascularized scaffold or a non-vascularized scaffold over 12 days. CD31 is an integral membrane protein that is expressed on the surface of endothelial cells and mediates cell-cell adhesion and interactions involving angiogenesis. VE cadherin is important for maintaining newly formed vessels. Increased VE cadherin and CD31 staining was observed for the pre-vascularized scaffolds as compared to the non-vascularized scaffolds, indicating conversion of the hMSCs into vascular endothelial cells (VECs).

Overall, these data indicated enhanced conversion of hMSCs into VECs and neovascularization when scaffolds were pre-vascularized to generate a pro-angiogenic matrix. Similar results are expected and have been observed with 3D-printed scaffolds made from the same PLA polymer material.

### Example 6

### Additional Micro Pore Formation in Scaffolds

Scaffolds must be highly porous in order to support bone formation and the formation of its vasculature. In addition to the interconnected network of spaces present in the trabecular core portion and the hollow channels present in the cortical exterior portion, additional pores are introduced using a soak-freeze technique. Scaffolds are first soaked in DI water overnight. After removing excess water, the scaffolds are frozen by placing them in liquid nitrogen or in the freezer at −80° C. overnight. To check for the presence and structure of soak-freeze induced pores, scanning electron microscopy (SEM) is used. In addition to this, liquid extrusion porosimetry (LEP-1100A, PMI, Ithaca, NY) is used to determine and compare the pore size distributions produced as a result of different freezing methods.

### Example 7

In vivo Testing of 3D Scaffolds

A New Zealand white rabbit critical-sized radial defect model was used to study the efficacy of bone regeneration over 20 weeks using a mineralized and pre-vascularized 3D scaffold including a trabecular core portion and a cortical exterior portion surrounding its circumference (Design 2 from Example 2 and FIG. 4B).

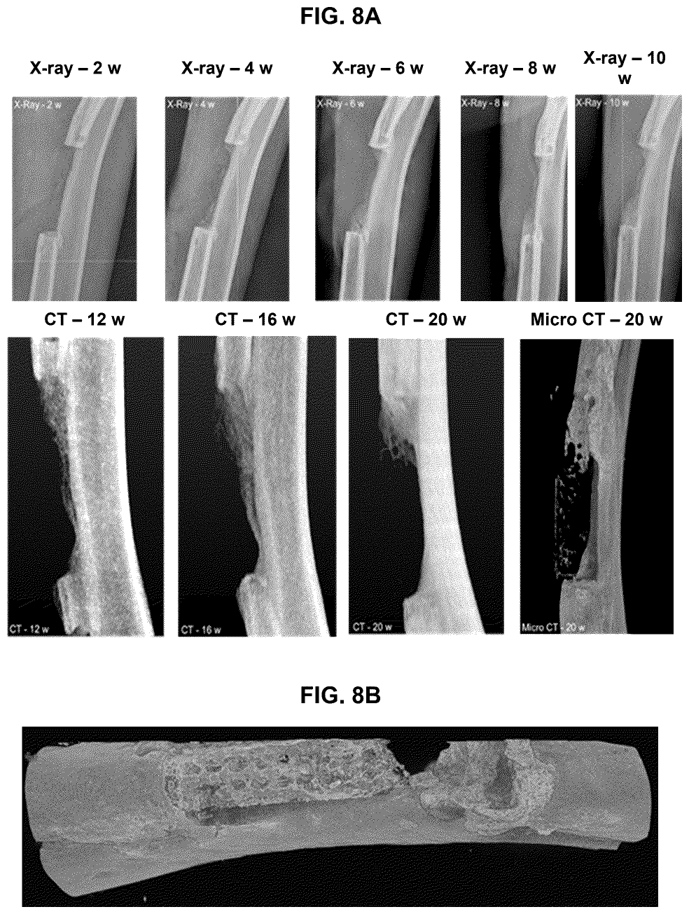

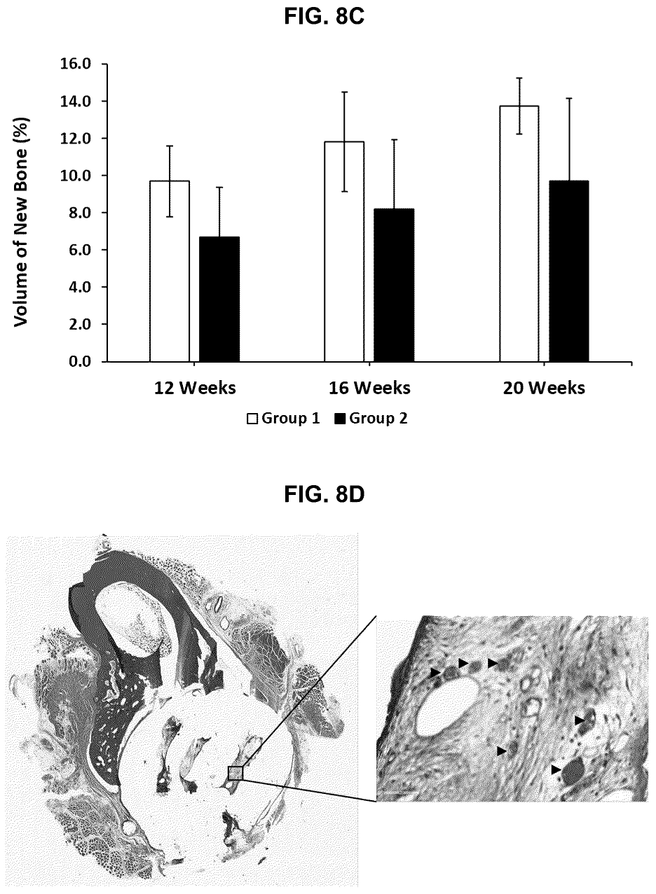

FIG. 8A-D show results of bone regeneration using this load-sharing model. A critical-sized defect is one that will not heal or regenerate on its own over the animal's lifespan. A defect of 15 mm in size was created in the radius of the rabbits and scaffolds were then implanted. The study included 3 treatment groups: Group 1: a mineralized and pre-vascularized Design 2 scaffold without bone marrow taken from the animal; Group 2: a mineralized and pre-vascularized Design 2 scaffold with bone marrow taken from the animal; and Group 3: an allograft control.

FIG. 8A (top) shows X-ray images taken at the 2-week, 4-week, 6-week, 8-week, and 10-week timepoints after the radial defect surgery. A gradual increase in callus formation and progressive bone regeneration was observed. FIG. 8A (bottom) shows live animal CT scan images at the 12-week, 16-week, and 20-week timepoints showing bone growth into and within the scaffold, and a micro-CT scan image (post excision) at the end of the study showing formation of mature bone tissue. Scaffolds maintained their mechanical strength and structural integrity during the study. FIG. 8B shows a reconstructed micro-CT scan image showing clear bone infiltration and mature bone formation within the scaffold implant.

FIG. 8C shows the volume of new bone growth throughout the study as determined from the 3D reconstructed CT scan images at the 12-week, 16-week, and 20-week timepoints. The values are represented as a % of the total volume of the defect. Although the % volume of new bone was greater in Group 1 without bone marrow, Group 2 with bone marrow displayed a larger amount of bone infiltration in the scaffold structure.

FIG. 8D shows histological H&E staining analysis showing bone regeneration within the polymer scaffold implant along with the formation of blood vessels (shown with black arrowheads). Overall, these in vivo results demonstrated that the 3D scaffolds can maintain their mechanical strength and structural integrity while promoting bone regeneration and show promise as bone graft alternatives. Further in vivo studies are conducted in a load-sharing large animal model (e.g., sheep) to compare the effects of the disclosed 3D scaffolds against the standard of care graft approaches.

### Example 8

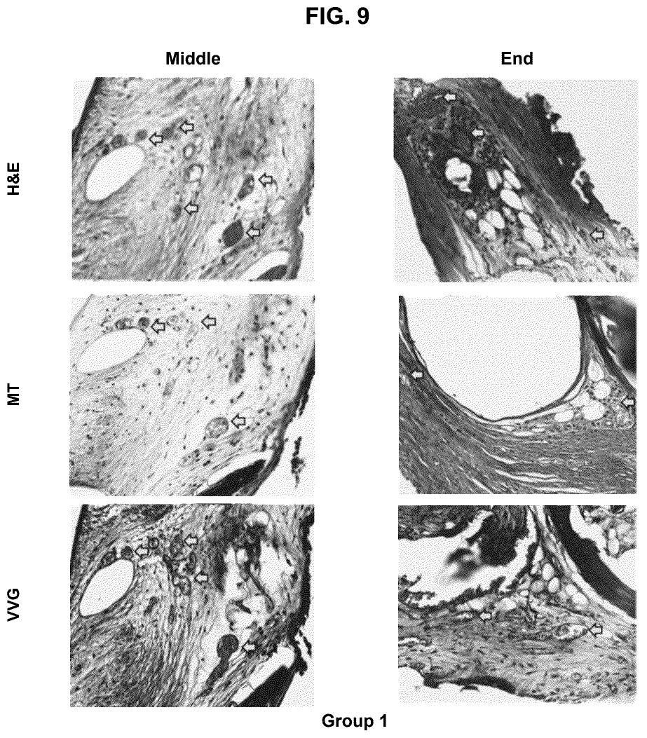

Histology Staining Sections from implanted scaffolds focusing on new bone tissue and blood vessels within the scaffold structure were stained. See FIG. 9-11. All images were obtained at high magnification (40×) and showed the regenerated bone tissue present within the scaffold. Mineralized bone tissue appeared pink (H&E), blue (MT), and red (VVG), while unmineralized bone tissue appeared light pink (H&E), light blue (MT), and light red/pink (VVG). Arrows indicate the presence of blood vessels containing erythrocytes, VVG staining also revealed elastic fibers in the vessel walls, stained black, confirming vascularization.

FIG. 9 shows histological staining of unseeded scaffolds. These samples were mineralized and prevascularized without adding autologous bone marrow. Arrows indicate blood vessels. Mineralized bone tissue appeared dark pink (H&E), blue (MT), and red (VVG).

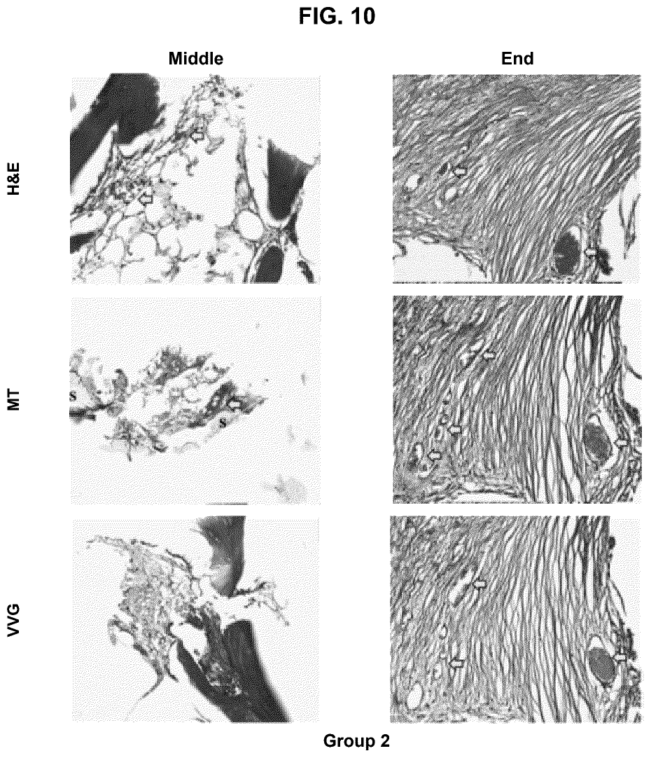

FIG. 10 shows histological staining of bone marrow seeded scaffolds. These samples were mineralized, prevascularized scaffolds seeded with autologous bone marrow. Mineralized bone tissue appeared dark pink (H&E), blue (MT), and red (VVG).

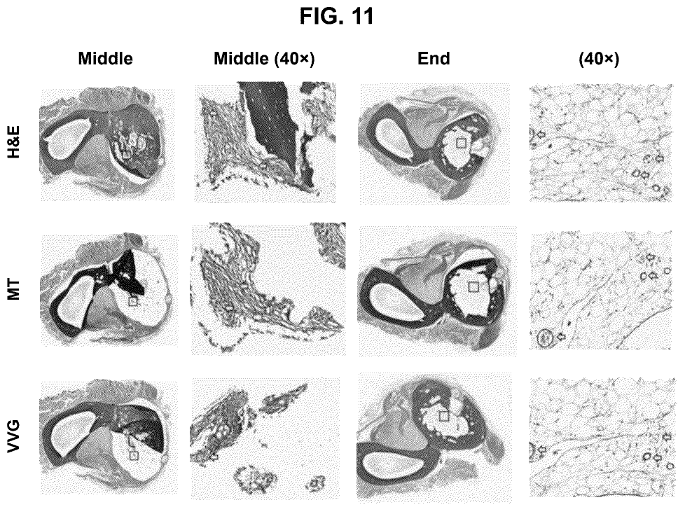

FIG. 11 shows histological staining of allografts. There is unmineralized and mineralized tissue. The tissue in the middle contained much smaller blood vessels than those seen in implants.

### Example 9

### Immunostaining

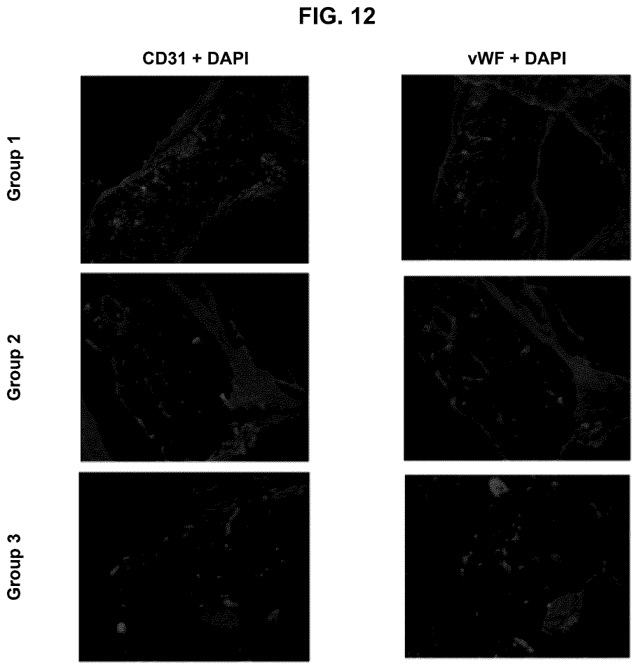

Samples were stained with anti-CD31 and anti-vWF antibodies which indicate blood vessel growth. All groups showed positive staining for both CD31 and VWF. See FIG. 12. The positively stained area in CD31 stained images was quantified as the percentage of positively stained area to total area. Data differed with location on implant (ends vs. middle). The greatest staining was observed in the seeded implants at ends and in the middle for allographs.

### Quantification of Cd31 Staining

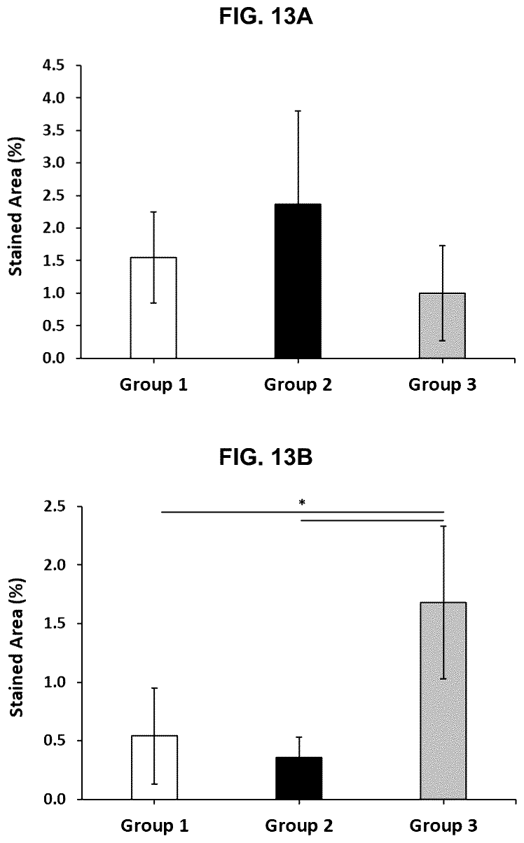

The percentage of the stained area for anti-CD31 (FIG. 11) was calculated and compared across the three groups, with analyses performed on sections from the middle of the implant (FIG. 13A) and the ends (FIG. 13B). * Indicates statistical significance with p<0.05. Seeded scaffolds had the highest amount of CD31 (not significant) at the ends of the implant. Allografts had the greatest amount in the middle.

1. A three-dimensional scaffold for bone repair or regeneration comprising:
a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and
an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion.

2. The scaffold of claim 1, wherein the interconnected network of spaces forms from about 45% to about 55% of the volume of the core portion.

3. The scaffold of claim 1, wherein each strut in each layer has a width ranging from about 0.4 mm to about 0.6 mm, and wherein each space positioned between adjacent struts in each layer has a width ranging from about 0.4 mm to about 0.6 mm.

4. The scaffold of claim 1, wherein the core portion and the exterior portion are each independently formed of a material comprising at least one biocompatible polymer selected from the group consisting of a polycarbonate, a polymethylmethacrylate, a polyethylene, a polyurethane, a polyaryl etherketone, a polyetherether-ketone, a polylactic acid (PLA), a polylactide, a polyglycolide, a poly(D, L-lactide), a poly(L-lactide), a poly(glycolide), a poly(s-caprolactone), a poly(dioxanone), a poly(glyconate), a poly(hydroxybutyrate), a poly(hydroxyvalerate), a poly(orthoester), a poly(carboxylate), a poly(propylene fumarate), a poly(phosphate), a poly(anhydride), a poly(iminocarbonate), a poly(phosphazene), and copolymers thereof.

5. (canceled)

6. The scaffold of claim 1, wherein the exterior portion has an exterior surface, and wherein the surfaces of the struts and the exterior surface of the exterior portion are coated with a calcium phosphate-based mineral comprising hydroxyapatite, alpha tricalcium phosphate, beta tricalcium phosphate, or combinations thereof.

7. (canceled)

8. The scaffold of claim 1, wherein one or more of the channels of the exterior portion are at least partially filled with a pro-angiogenic extracellular matrix.

9. The scaffold of claim 1, wherein each of the channels of the exterior portion has a cross-sectional width ranging from about 0.25 mm to about 2.0 mm or from about 0.5 mm to about 1.0 mm.

10. (canceled)

11. The scaffold of claim 1, wherein each of the channels of the exterior portion is spaced apart from adjacent channels by about 0.5 mm to about 1.0 mm.

12. he scaffold of claim 1, wherein the core portion has a diameter ranging from about 3 mm to about 24 mm.

13. The scaffold of claim 1, wherein the scaffold has a diameter ranging from about 5 mm to about 35 mm, or wherein a length of the scaffold from the first end to the second end ranges from about 5 mm to about 500 mm.

14. (canceled)

15. The scaffold of claim 1, wherein the scaffold has a compressive modulus ranging from about 400 MPa to about 700 MPa, or wherein the scaffold has a compressive yield strength ranging from about 20 MPa to about 50 MPa.

16. (canceled)

17. The scaffold of claim 1, further comprising mesenchymal stem cells (MSCs).

18. (canceled)

19. The scaffold of claim 1, wherein the scaffold is three dimensionally printed.

20. A method of making a three-dimensional scaffold for bone repair or regeneration, the method comprising:
fabricating the three-dimensional scaffold comprising:
a substantially cylindrical core portion comprising a plurality of substantially circular layers stacked on each other, the core portion having a longitudinal axis orthogonal to and extending through a center of each substantially circular layer, each layer having a plurality of struts spaced apart from each other and arranged substantially parallel to each other so as to define a plurality of spaces, each space positioned between adjacent struts in the layer, wherein the struts and spaces of each layer are substantially orthogonal to the struts and spaces of adjacently stacked layers such that all the spaces within the core portion define an interconnected network of spaces extending through the plurality of layers, wherein the interconnected network of spaces forms from about 40% to about 60% of the volume of the core portion; and
an exterior portion surrounding a circumference of the core portion and including a plurality of channels, each of the channels being substantially parallel to the longitudinal axis of the core portion and extending through the exterior portion from a first end of the scaffold to a second end of the scaffold, wherein the plurality of channels forms from about 20% to about 30% of the volume of the exterior portion;
coating the surfaces of the struts and an exterior surface of the exterior portion with a calcium phosphate-based mineral to mineralize the scaffold;
seeding and culturing cells in one or more of the channels of the exterior portion for a period of time sufficient to produce a pro-angiogenic extracellular matrix that at least partially fills the one or more channels; and
removing the cells from the one or more channels of the exterior portion.

21. The method of claim 20, wherein fabricating the three-dimensional scaffold comprises three-dimensionally printing the scaffold.

22. The method of claim 20, wherein the calcium phosphate-based mineral comprises hydroxyapatite, alpha tricalcium phosphate, beta tricalcium phosphate, or combinations thereof.

23. The method of claim 20, wherein the cells comprise endothelial cells.

24. The method of claim 20, further comprising one or more of sterilizing the scaffold, freeze-drying the scaffold, adding stem cells to the scaffold, or adding autologous bone marrow to the scaffold.

25-27. (canceled)

28. A method of repairing or regenerating bone in a subject, the method comprising implanting the scaffold of claim 1 into a site in need of bone repair or regeneration in the subject.

29. A kit for repairing or regenerating bone in a subject, the kit comprising:
the scaffold of claim 1; and
one or more of packaging or instructions for use.
